(19) United States
(12) Patent Application Publication
(10) Pub. No.: US 2026/0183167 A1
(43) Pub. Date: Jul. 2, 2026

(54) SHOULDER TRACTION DEVICE FOR CERVICAL SPINE SURGERY POSITIONING

(71) Applicant: Craig D. STEINER, Hollywood, FL (US)

(72) Inventor: Craig D. STEINER, Hollywood, FL (US)

(21) Appl. No.: 19/425,500

(22) Filed: Dec. 18, 2025

Related U.S. Application Data
(60) Provisional application No. 63/741,051, filed on Jan. 1, 2025.

(51) Int. Cl. A61G 13/00; A61B 6/04; A61G 13/12

(52) U.S. Cl. CPC A61G 13/0054; A61B 6/0407; A61G 13/1255; A61G 13/126; A61G 13/1295; A61G 2200/54; A61G 2210/10

(57) ABSTRACT

Provided is a traction-setting device for an individual undergoing spine-related procedures. Straps are used to enable a medical practitioner to increase traction on the shoulder of a patient by means of a loose strap that can be pulled in the upward direction, whilst a second strap is pulled in the downward direction to enable further traction. The traction device is adjustable allowing for easy and reproducible modifications to the amount of traction with minimal effort and disruption during pre-operative positioning and in the event of intraoperative needs. Corresponding methods are also disclosed herein.

### CROSS-REFERENCE TO RELATED APPLICATIONS

This application claims the benefit of U.S. Provisional Patent Application No. 63/741,051, filed Jan. 1, 2025, which is hereby incorporated by reference.

### FIELD OF THE INVENTION

The presently disclosed subject matter relates to the field of traction devices for spine-related procedures such as surgery or x-ray imaging. Straps are used to enable a medical practitioner to increase traction at the shoulder of a patient by means of a loose strap that can be pulled in the upward direction, whilst a second strap is pulled in the downward direction to enable further traction. Corresponding methods are also disclosed herein.

### BACKGROUND

When performing cervical spine surgery, it is important to be able to obtain intraoperative x-rays for many reasons including confirming the levels of surgery, placement of hardware, instrument localization, spinal alignment, among others. Obtaining an anteroposterior image or AP image is simple enough using a radiolucent table.

Obtaining the lateral image is more difficult. In the prone or supine position, the shoulders will often obscure various levels of the cervical spine. Therefore, as part of positioning for both anterior and posterior cervical procedures, it is standard practice to utilize traction to pull the shoulders down toward the feet to enable optimal imaging of the cervical spine in the lateral position.

There are several positioning systems that are available which all require changes and alterations to the traditional set up and positioning. These include arm wraps, pads that go in front and back of the chest and over the tops of the shoulders, foam positioning devices, wrist restraints, among others.

The most common method for shoulder traction includes using tape. After the arms are tucked, tape is applied to the shoulder and pulled down and wrapped typically to the foot of the rail of the operating table.

Additionally, wrist restraints are commonly attached to the wrists for additional traction when needed.

The cervical spine surgery shoulder positioning device which is the subject of this patent includes an adhesive patch that is applied to the top of the shoulder and the proximal aspect of the arm which is attached to a strap system which provides the ability to quickly and easily adjust the traction and lock it in place, release traction and reset easily and rapidly, and provides the ability to apply secondary manual traction if necessary.

### SUMMARY

In one embodiment, the invention provides a traction device for a spine-related procedure in an individual, the traction device comprising:
a traction-setting strap comprising an adhesive pad at a first end of the traction-setting strap wherein the adhesive pad is configured to adhere to a shoulder and proximal aspect of the arm of the individual;
a fixed strap configured to attach to an operating room table;
a first buckle attached to the fixed strap and wherein the first buckle is further configured such that the tension-setting strap passes through it and is configured to adjust the traction by means of pulling on the loose end of the traction-setting strap upwards towards the head of the individual; and
a second strap attached to the traction-setting strap, wherein the second strap comprises a traction loop, the second strap configured to provide additional means of traction when pulled, to enable the traction-setting strap to be tightened when the traction device is secured on the individual.

In one embodiment, the adhesive pad comprises removable adhesive, Velcro, or a combination thereof. In one embodiment, the adhesive pad further comprises an affixing loop configured to secure around the shoulder of the individual. In one embodiment, the adhesive pad is modular and configured to be detachable. In one embodiment, the traction device further comprises a second buckle affixed on the second strap and configured to secure the second strap in place by means of pulling on the loose end of the second strap.

In one embodiment, the first buckle, the second buckle, or a combination thereof, is a ratchet buckle.

In one embodiment, the first buckle is reversible.

In one embodiment, the traction-setting strap, fixed strap and the second strap comprise any of the following selected from: polyester webbing, nylon webbing, rubber, polypropylene, synthetic fiber, or a combination thereof.

In one embodiment, the spine-related procedure is any of the following selected from: diagnostic procedure, imaging procedure, interventional procedure, and surgery.

In one embodiment, the invention provides a traction device for a spine-related procedure in an individual, the traction device comprising:
a fixed strap comprising an adhesive pad at a first end of the fixed strap wherein the adhesive pad is configured to adhere to a proximal aspect of the arm of the individual;
a traction-setting strap with one end configured to attach to an operating room table;
a first buckle attached to the fixed strap and wherein the first buckle is further configured such that the tension setting strap passes through it and is configured to adjust the traction by means of pulling on the loose end of the traction-setting strap downwards towards the feet of the individual; and
a second strap attached to the fixed strap, wherein the second strap comprises a traction loop, the second strap configured to provide additional means of traction when pulled, to enable the traction-setting strap to be tightened when the traction device is secured on the individual.

In one embodiment, the invention provides a traction device for a spine-related procedure in an individual, the traction device comprising:
a first traction-setting strap comprising an adhesive pad at a first end of the first traction-setting strap wherein the adhesive pad is configured to adhere to a proximal aspect of the arm of the individual;
a second traction-setting strap with one end configured to attach to an operating room table;
a first buckle attached to the first traction-setting strap and wherein the first buckle is further configured such that the first traction-setting strap passes through it and is configured to adjust the traction by means of pulling the first traction-setting strap upwards towards the head of the individual; and wherein the first buckle is further configured to attach to the second traction-setting strap through which the second traction-setting strap passes and is configured to adjust the traction by means of pulling the second traction-setting strap downwards towards the feet of the individual;
a third strap attached to the first traction-setting strap, wherein the third strap comprises a traction loop, wherein the third strap is configured to provide additional means of traction when pulled, to enable the first traction-setting strap, the second traction-setting strap, or a combination thereof, to be tightened when the traction device is secured on the individual.

In one embodiment, the invention provides a method of positioning an individual for a spine-related procedure, the method comprising:
providing the traction device of any one of the previous claims;
applying the adhesive patch to the shoulder and/or proximal aspect of the arm of the individual;
affixing the fixed strap to the operating room table; and
pushing the proximal aspect of the shoulder and/or arm towards the foot and pulling the traction-setting strap.

In one embodiment, the method further comprises pulling the second strap before pulling the traction-setting strap, to achieve higher traction.

In one embodiment, the method further comprises restraining the arms of the individual before securing the traction device.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is a schematic illustration of the traction-device in one embodiment of the invention, configured to provide traction by pulling the traction-setting strap upwards.

FIG. 2 is a schematic illustration of the traction-device in one embodiment of the invention, configured to provide traction by pulling the traction-setting strap downwards.

FIG. 3 is a schematic illustration of the traction-device in one embodiment of the invention, configured to provide traction by pulling two traction-setting straps upwards and downwards.

FIG. 4 is a schematic illustration of a detachable adhesive pad connected to a strap by an attachment means.

For simplicity and clarity of illustration, elements shown in the figures are not necessarily drawn to scale, and the dimensions of some elements may be exaggerated relative to other elements. In addition, reference numerals may be repeated among the figures to indicate corresponding or analogous elements.

### DETAILED DESCRIPTION

The disclosure provided herein relates to a traction device for a spine-related procedure in an individual. One aim of the traction device is to enable better positioning of the individual's body, or any part of the body, for the spine-related procedure that will be carried out. In particular, the presently disclosed traction device allows a practitioner who is applying or setting up the device on the person to improve traction. As used herein “traction” refers to the application of a pulling force to a part of the body to achieve a specific outcome such as aligning, stabilizing, or positioning a structure. For example, in a shoulder-positioning traction device in spine-related procedures, traction would involve pulling the shoulder (or arm) in a specific direction (e.g., downward, upward, or laterally) to position the spine or surrounding structures for surgical access or imaging. Thus, a traction-device is used to apply traction to pull the shoulder downward to open up the cervical or thoracic area for better surgical access or visualization on imaging. Traction is correlated with tension in the strap-based traction-device. As used herein “tension” refers to the internal force within the strap/traction-device itself due to being stretched or tightened. It is the resistance or force generated within the material of the strap. For example, the practitioner adjusts how tight the strap/s is/are to ensure it holds the shoulder firmly in place. Positioning a traction-device correctly allows for enhanced surgical access, improved patient positioning, consistent and controlled force, decrease tissue damage, improve surgical outcome, enhance safety in the individual/patient, enhanced safety for operating room staff and support personnel, versatility, time efficiency, etc.

In one embodiment, the invention provides a traction device for a spine-related procedure in an individual, the traction device comprising:
a traction-setting strap comprising an adhesive pad at a first end of the traction-setting strap wherein the adhesive pad is configured to adhere to a shoulder and proximal aspect of the arm of the individual;
a fixed strap configured to attach to an operating room table;
a first buckle attached to the fixed strap and wherein the first buckle is further configured such that the tension-setting strap passes through it and is configured to adjust the traction by means of pulling on the loose end of the traction-setting strap upwards towards the head of the individual; and
a second strap attached to the traction-setting strap, wherein the second strap comprises a traction loop, the second strap configured to provide additional means of traction when pulled, to enable the traction-setting strap to be tightened when the traction device is secured on the individual.

As used herein the “traction-device” refers to a device to improve traction in an individual. The individual being any person that wears the traction-device; otherwise referred to as ‘patient’ herein.

The “traction-setting” strap refers to a strap that regulates the tension in the strap to achieve a specific traction. This is typically affixed to the patient wherein the strap is pulled tight (e.g., via a buckle) and held at a particular tension. A secondary (also referred to as “second”) strap can be separately pulled to achieve additional tension/traction either for temporary increased traction or to enable the traction setting strap can be set at a higher force.

The adhesive pad can be placed on any part of the arm, but is typically located at a particular point on, or around the shoulder and onto the proximal arm. In one embodiment, the adhesive pad comprises a removable adhesive, Velcro, or a combination thereof. In one embodiment the adhesive substance is a glue. In one embodiment, the adhesive pad comprises a removable adhesive. In various embodiments, the adhesive pads comprise medical adhesive pads, peelable adhesives, pressure-sensitive adhesives (PSAs), medical-grade adhesives, acrylic-based removable adhesives, etc. The adhesive pads used possess the strong adhesion required for the high traction applications in spine-surgery. The removal of such pads is typically painless and leaves no permanent marks. In one embodiment, the adhesive pad further comprises a release liner; otherwise referred to as a protective backing.

In one embodiment, the adhesive pad further comprises an affixing loop configured to secure around the shoulder of the individual. Therefore, in addition to a regular adhesion by means of a removeable adhesive, this additional component further secures the individual's arm/shoulder in place.

In one embodiment, the adhesive pad is modular and configured to be detachable. In one embodiment, the adhesive pad is disposable. As such, the adhesive pad is attached to the traction-setting strap by a connecting means. This connecting means can be a hinge, a button, Velcro strip, magnetic fasteners, snap buttons, clips, quick release clasp, rivets, zipper, elastic bands, hook-and-loop fastener, press studs, etc. One advantage of this modular assembly is that it enables the adhesive pad to be easily replaced for hygienic use between patients, in accordance with regulatory requirements.

As used herein the “proximal aspect of the arm” refers to the part of the upper arm that is closer to the shoulder as opposed to closer to the elbow.

As used herein the “traction loop” refers to a loop that enables the traction-device to be secured to a structure. Typically, the traction loop is affixed to an operating room table e.g., to the rail, table post, chair, etc. In one embodiment the traction loop is a loop which is large enough to secure on to the structure that it is suited for. Otherwise, the traction loop can be formed by a loop secured by a Velcro strap. Affixing means refers to the method by which they affix on the (e.g.,) operating room table. For example, a whole-loop, a Velcro-comprising adjustable loop. In one embodiment the traction-loop is a size-adjustable loop.

In one embodiment, the first buckle affixes on the traction-setting strap and is configured to secure the strap in place by means of pulling on the loose end of the traction-setting strap. This buckle provides the means by which to set the tension of the strap.

In one embodiment, the second strap is attached to the traction-setting strap, wherein the second strap comprises a traction loop, the second strap configured to provide additional means of traction when pulled, to enable temporary additional traction or to enable the traction-setting strap to be tightened when the traction device is secured on the individual.

In one embodiment, the traction-device further comprises a second buckle affixed on the second strap and configured to secure the second strap in place by means of pulling on the loose end of the second strap.

In one embodiment, the first buckle, the second buckle, or a combination thereof, is a ratchet buckle. Other types of buckles include, but are not limited to: ratchet buckle, side-release buckle, push-button buckle, tongue buckle, roller buckle, cam buckle, G-hook buckle, magnetic buckle, D-ring buckle, Velcro buckle, quick-release buckle, hook-and-loose buckle, etc.

Typically, such strap devices only allow for pulling ‘down’ towards the legs of the individual. However, practitioners often require positioning the shoulder in place at the time of applying force/traction/tension to the traction-device for positioning. In one embodiment of the currently described device, the first buckle can be configured such that the practitioner that is tightening the traction device on the individual can tighten the traction-setting strap whilst standing in the proximity of the head of the individual. This allows the same practitioner to apply the manual traction with direct downward force on the shoulder with one hand while tightening and setting the traction with the other hand pulling up toward the head on the traction setting strap. In one embodiment the first buckle is configured such that the pulling is in the direction of the legs of the individual. In one embodiment the first buckle is reversible.

In one embodiment, the traction-setting strap, fixed strap and the second strap comprise any of the following: strapping or webbing made from polyester, polypropylene, nylon, rubber, synthetic fibers or materials, or combination thereof. The width of any of the straps described herein is selected from: about 1 inch, about 2 inches, about 3 inches, about 4 inches, about 5 inches, or a combination thereof. For example, the traction-setting strap can be 2 inches wide, whereas the second strap is 3 inches wide.

As described herein, the traction-device of the present invention is utilized in any spine-related procedure. Examples of spine-related procedures include, but are not limited to: diagnostic procedure, imaging procedure, interventional procedures, and surgery. An example of an imaging procedure is an x-ray. Examples of interventional procedures include but are not limited to injections and biopsies. Examples of surgery include but are not limited to cervical spinal endoscopy, anterior cervical discectomy and fusion, disc replacement surgery, cervical laminectomy, posterior spinal fusion, and placement of spinal instrumentation.

The invention provides a method of positioning an individual for a spine-related procedure, the method comprising:
providing the traction device of the invention;
applying the adhesive patch to the shoulder and/or proximal aspect of the arm of the individual;
affixing the fixed strap to the operating room table; and
pushing the proximal aspect of the shoulder and/or arm towards the foot and pulling the traction-setting strap.

The positioning of the individual can be carried out before, during, or after the spine-related procedure, or a combination thereof. In one embodiment the method further comprises pulling the second strap before pulling the traction-setting strap, to achieve higher traction. The pulling of the traction-setting strap can be in the upwards direction (i.e., towards the head of the patient) or in the downward direction (i.e., towards the feet of the patient), depending on the configuration of the buckle.

In one embodiment, a force-meter is further incorporated into the traction-setting strap, or the second strap, or the fixed strap, and is configured to measure the force applied on the strap. Although each patient's positioning requirements may be different, the force-meter enables a consistent use of force for the procedures, as required.

In one embodiment, the method further comprises restraining the arms of the individual before securing the traction device. The restraining can be achieved by swaddling the individual with a sheet thereby restraining the arms. In one embodiment, the traction device comprises a restraining strap attached to the traction device wherein the restraining strap is configured to restrain the arms of the individual.

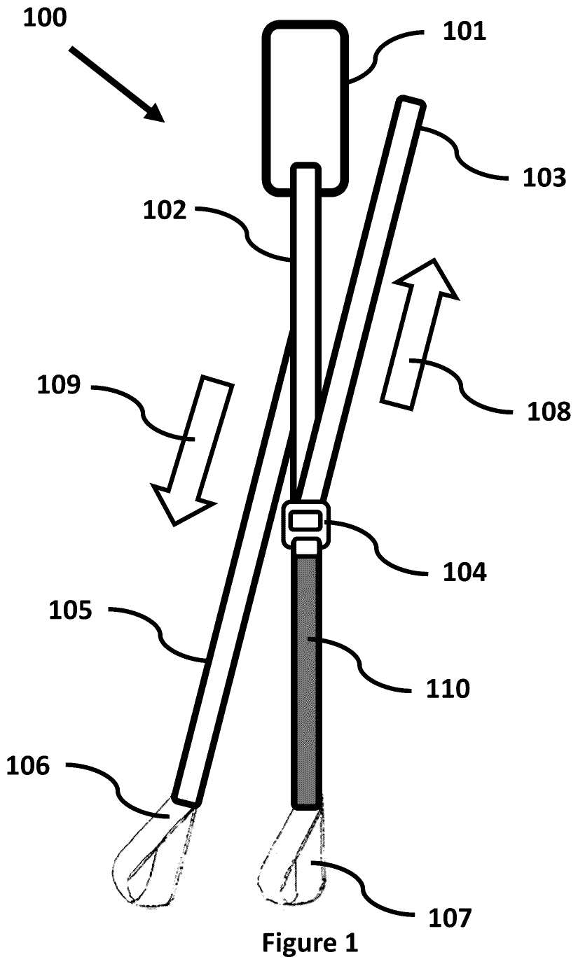

FIG. 1 shows one embodiment of the traction-device 100 of the invention wherein the traction-setting strap 102 has an adhesive pad 101 at one end which is affixed to the shoulder (not shown). The loose end of the traction setting strap 103 passes through the first buckle 104. The first buckle 104 is secured to the fixed strap (grey) 110, which is itself secured to the operating room table by the loop 107. The loop 107 is shown as a closed loop but can also be size adjustable with Velcro, or open/closeable. The loose end 103 of the strap can be pulled upwards 108 (arrow) in the general direction of the adhesive pad 103, in close proximity to the head of the individual. This is advantageous since often the shoulder of the patient needs to be positioned/pushed/directed at the same time that the traction/tension of the strap needs to be applied.

Additionally, FIG. 1 shows the second strap 105 attached to the upper section of the tension-setting strap 102. The second strap 105 is pulled downwards 109 to increase traction on the shoulder. Pulling the second strap 105 downwards 109 (arrow) can be done for temporary increased manual traction or to enable the traction-setting strap 102 to be pulled tighter by pulling the loose end 103 through the buckle. Although the second strap 105 is shown to be positioned above the first buckle 104 on the traction-setting strap 102, it can be located below the first buckle 104, in other embodiments. In one embodiment, the second strap 105 further comprises a second buckle (not shown) to further enable an increased tension. In other embodiments, additional straps can be attached onto the traction-device to further enable traction, as required.

The traction-device 100 is typically designed for use on one shoulder of a patient. Therefore, if a patient were to require both shoulders to be traction-strapped, two separate traction devices would be used to achieve this result. The traction devices for each shoulder being operated independently.

In one embodiment, the traction-device 100 incorporates two adhesive pads 101 (second adhesive pad not shown) separated by a split/fork in the traction-setting strap 101, the adhesive pads to be placed on both shoulders, wherein the single traction-setting strap 102 is used to achieve the traction for both shoulders.

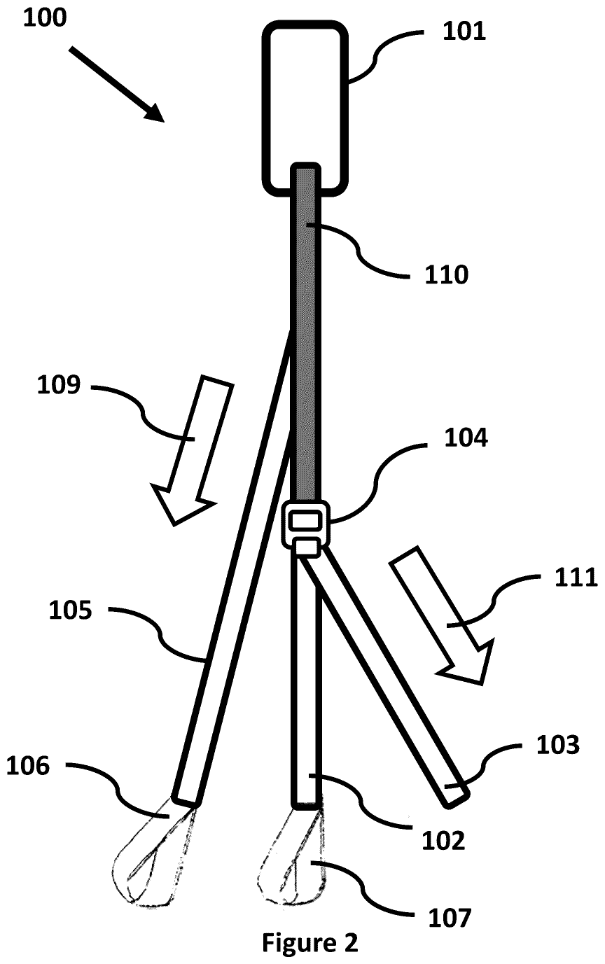

An alternative configuration is provided which enables the practitioner to secure the traction device on the patient by pulling downwards on the traction-setting strap, towards the feet of the individual. FIG. 2 shows a traction device 100 wherein a fixed strap (grey) 110 is attached to an adhesive pad 101. The fixed strap (grey) 110 is attached to a buckle 104. The traction-setting strap 102 attaches to a securing loop 107 wherein the loose end 103 of the traction-setting strap passes through the buckle 104 such that the traction-setting strap 103 can be pulled downwards 111 towards the feet of the individual (not shown).

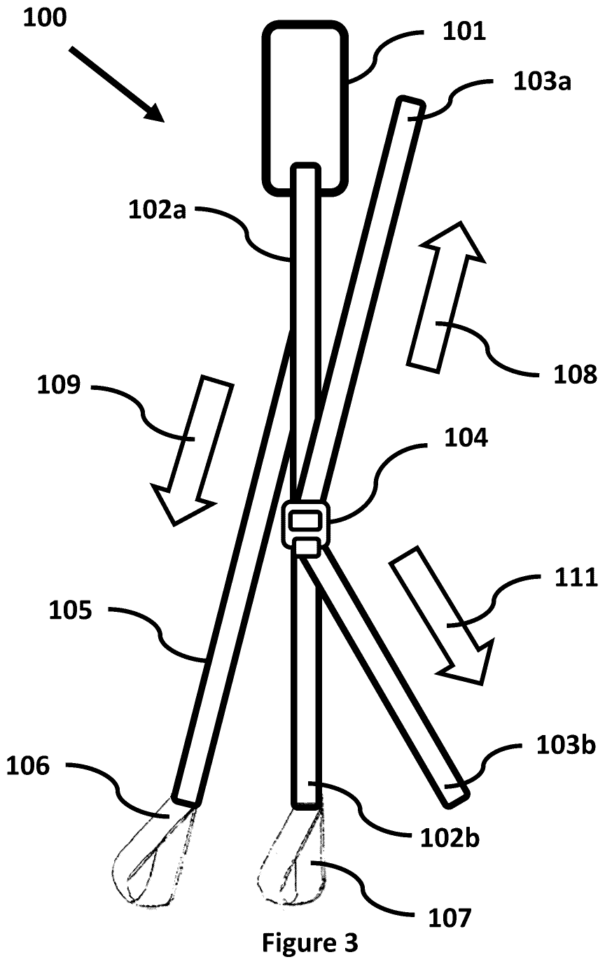

A further configuration is provided where two traction-setting straps are configured to enable the traction by pulling both downwards towards the feet of the individual and upwards towards the head of the individual by means of two separate straps. FIG. 3 shows a first traction-setting strap 102a attached to the adhesive pad 101 and a second traction-setting strap 102b with a securing loop 107 at one end. The first traction-setting strap 102a and the second traction-setting strap 102b pass through a buckle 104 wherein the loose end 103a of the first traction-setting strap 102 a is configured to be pulled upwards 108 and the loose end 103b of the second traction-setting strap 102b is configured to be pulled downwards 111. The additional strap (otherwise referred to as the second or third strap, depending on the configuration) 105 is pulled downwards 109 to increase traction on the shoulder. Pulling the second strap 105 downwards 109 (arrow) can be done for temporary increased manual traction or to enable the traction-setting straps 102 to be pulled tighter by pulling on the loose ends 103a and 103b.

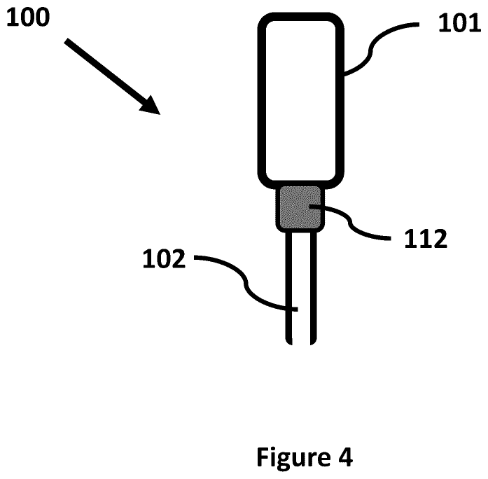

FIG. 4 shows part of the traction device 100 where the adhesive pad 101 is disposable and is attached by an attaching means 112, such as a fast release clip, to one of the straps 102 of the traction device 100. Examples for the attaching means 112 include, but are not limited to: a hinge, a button, Velcro strip, magnetic fasteners, snap buttons, clips, quick release clasp, rivets, zipper, elastic bands, hook-and-loop fastener, press studs, buckle, etc.

In one embodiment, the term “a” or “one” or “an” refers to at least one. In one

embodiment the phrase “two or more” may be of any denomination, which will suit a particular purpose. In one embodiment, “about” or “approximately” may comprise a deviance from the indicated term of +1 %, or in some embodiments, −1 %, or in some embodiments, ±2.5 %, or in some embodiments, ±5 %, or in some embodiments, ±7.5 %, or in some embodiments, ±10 %, or in some embodiments, ±15 %, or in some embodiments, ±20 %, or in some embodiments, ±25 %.

Those skilled in the art to which this invention pertains will readily appreciate that numerous changes, variations, and modifications can be made without departing from the scope of the presently disclosed subject matter, mutatis mutandis.

1. A traction device for a spine-related procedure in an individual, the traction device comprising:
a traction-setting strap comprising an adhesive pad at a first end of the traction-setting strap wherein the adhesive pad is configured to adhere to a shoulder and proximal aspect of the arm of the individual;
a fixed strap configured to attach to an operating room table;
a first buckle attached to the fixed strap and wherein the first buckle is further configured such that the tension-setting strap passes through it and is configured to adjust the traction by means of pulling on the loose end of the traction-setting strap upwards towards the head of the individual; and
a second strap attached to the traction-setting strap, wherein the second strap comprises a traction loop, the second strap configured to provide additional means of traction when pulled, to enable the traction-setting strap to be tightened when the traction device is secured on the individual.

2. The traction device of claim 1 wherein the adhesive pad comprises removable adhesive, Velcro, or a combination thereof.

3. The traction device of claim 1 wherein the adhesive pad further comprises an affixing loop configured to secure around the shoulder of the individual.

4. The traction device of claim 1 wherein the adhesive pad is modular and configured to be detachable.

5. The traction device of claim 1 further comprising a second buckle affixed on the second strap and configured to secure the second strap in place by means of pulling on the loose end of the second strap.

6. The traction device of claim 5 wherein the first buckle, the second buckle, or a combination thereof, is a ratchet buckle.

7. The traction device of claim 1 wherein the first buckle is reversible.

8. The traction device of claim 1 wherein the traction-setting strap, fixed strap and the second strap comprise any of the following selected from: polyester webbing, nylon webbing, rubber, polypropylene, synthetic fiber, or a combination thereof.

9. The traction device of claim 1 wherein the spine-related procedure is any of the following selected from: diagnostic procedure, imaging procedure, interventional procedure, and surgery.

10. A traction device for a spine-related procedure in an individual, the traction device comprising:
a fixed strap comprising an adhesive pad at a first end of the fixed strap wherein the adhesive pad is configured to adhere to a proximal aspect of the arm of the individual;
a traction-setting strap with one end configured to attach to an operating room table;
a first buckle attached to the fixed strap and wherein the first buckle is further configured such that the tension setting strap passes through it and is configured to adjust the traction by means of pulling on the loose end of the traction-setting strap downwards towards the feet of the individual; and
a second strap attached to the fixed strap, wherein the second strap comprises a traction loop, the second strap configured to provide additional means of traction when pulled, to enable the traction-setting strap to be tightened when the traction device is secured on the individual.

11. A traction device for a spine-related procedure in an individual, the traction device comprising:
a first traction-setting strap comprising an adhesive pad at a first end of the first traction-setting strap wherein the adhesive pad is configured to adhere to a proximal aspect of the arm of the individual;
a second traction-setting strap with one end configured to attach to an operating room table;
a first buckle attached to the first traction-setting strap and wherein the first buckle is further configured such that the first traction-setting strap passes through it and is configured to adjust the traction by means of pulling the first traction-setting strap upwards towards the head of the individual; and wherein the first buckle is further configured to attach to the second traction-setting strap through which the second traction-setting strap passes and is configured to adjust the traction by means of pulling the second traction-setting strap downwards towards the feet of the individual;
a third strap attached to the first traction-setting strap, wherein the third strap comprises a traction loop, wherein the third strap is configured to provide additional means of traction when pulled, to enable the first traction-setting strap, the second traction-setting strap, or a combination thereof, to be tightened when the traction device is secured on the individual.

12. A method of positioning an individual for a spine-related procedure, the method comprising:
providing the traction device of any one of the previous claims;
applying the adhesive patch to the shoulder and/or proximal aspect of the arm of the individual;
affixing the fixed strap to the operating room table; and
pushing the proximal aspect of the shoulder and/or arm towards the foot and pulling the traction-setting strap.

13. The method of claim 12 further comprising pulling the second strap before pulling the traction-setting strap, to achieve higher traction.

14. The method of claim 12 further comprising restraining the arms of the individual before securing the traction device.
